(19) United States
(12) Patent Application Publication
(10) Pub. No.: US 2003/0069639 A1
(43) Pub. Date: Apr. 10, 2003

(54) Methods and compositions for repair or replacement of joints and soft tissues

(76) Inventors: Tom Sander, Gainesville, FL (US); Russell S. Donda, Gainesville, FL (US); Christopher A. Seid, Gainesville, FL (US); Chester E. Sutterlin, Gainesvile, FL (US)

Correspondence Address: Donald J. Pochopien, McAndrews, Held & Malloy, Ltd., Citicorp Center, 34th Floor, 500 West Madison Street, Chicago, IL, 60661, US

(21) Appl. No.: 10/123,033

(22) Filed: Apr. 15, 2002

Related U.S. Application Data
(60) Provisional application No. 60/283,891, filed on Apr. 14, 2001.
(60) Provisional application No. 60/288,961, filed on May 6, 2001.
(60) Provisional application No. 60/328,283, filed on Oct. 9, 2001.

(51) Int. Cl. A61F 2/44

(52) U.S. Cl. 623/17.11

(57) ABSTRACT

Disclosed herein are methods and implants for enhancing or restoring the mechanical function of collagenous tissue. Specifically exemplified is the replacement or repair of endogenous nucleus pulposus with allogenic, xenogenic, or both nucleus pulposus that has been augmented with growth factors and glycosaminoglycans, via injection into a weakened intervertebral disc. Also disclosed is an implant to restore mechanical function to a damaged vertebral column. Additionally, methods and products for augmenting the extracellular matrix and cell content of a damaged nucleus pulposus through infusion of selected stem cells and other restorative materials are disclosed. The methods and products disclosed may be adapted for use in repair of all soft or hard tissue found in association with articulating joints.

### CROSS REFERENCE TO RELATED APPLICATION

This application claims the benefit under 35 USC§119(e) of Provisional Application No.: 60/283,891 filed Apr. 14, 2001, of Provisional Application No.: 60/288,961 filed May 6, 2001 and of Provisional Application No.: 60/328,283 filed Oct. 9, 2001.

### FIELD OF THE INVENTION

This invention is related generally to repair and replacement of collagenous tissue, and specifically to repair or replacement of damaged intervertebral disc and articular cartilage.

### BACKGROUND

As the average human life expectancy continues to increase in this country and worldwide, age related changes in collagenous tissue are becoming more and more prevalent. These changes, manifest in the stiffening of joints, deterioration of intervertebral discs, decreased elasticity of the vascular system and other disorders. Over time collagenous tissue gradually loses its ability to self-repair. Thus, damage caused by injury or degenerative disease will leave a permanent affect on its physiology and function. For example, degenerative disc disease (DDD), results in the loss of structure and function of the nucleus pulposus, the shock-absorbing center of spinal discs. With age, the initially soft and gelatinous nucleus pulposus is replaced by fibrocartilage. As the nucleus dehydrates and shrinks, the load on the nucleus decreases and the load on the annulus (the portion of the spinal disc that contains the nucleus pulposus) increases. Radial tears, cracks and fissures occur first within the annulus. If healing does not occur, the nucleus may migrate from the center of the disc to the periphery through the tear and compress a nerve. As the nucleus pulposus begins to dry out, its effectiveness as a “shock absorber” is reduced. As this protection is lost, the simple “wear and tear” of everyday activity can cause the vertebrae to develop jagged edges, called bone spurs, which can also compress nerves. Loss of intervertebral disc space due to a disc with diminished cushioning capacity can also cause nerve compression in the neuroforamen resulting in intense pain, often requiring surgical intervention. Traditionally, this problem has been corrected by percutaneous nucleotomy, chemonucleolysis, and laser disc decompression designed to accelerate disc degeneration and decrease pressure on adjacent nerves. In many cases this necessitates subsequent spinal fusion. However, this procedure fail to preserve spinal mobility and often leads to degeneration of adjacent discs (Matsuzaki, H. et al Spine 1996 21(2):178-183; Lee. C. K. Spine 1988(13): 375-77. Other reparative treatments have involved injection of polymers or other substances into the disc space to replace the nucleus pulposus damaged with age. For example, U.S. Pat. No. 6,206,921 discloses a method of replacing a damaged nucleus pulposus with a resilient synthetic material that will not disperse upon setting. Neither of these protocols provide complete relief from age related deterioration of the disc. Damage to collagenous tissue found in association with articulating bones is particularly problematic. Painful inflammation of a joint may result as cartilage that serves as a natural buffer between bone, becomes brittle and non-functional. Cumulative affects often manifest themselves in disabling diseases such as, for example, Arthritis or Osteoarthritis.

Until recently, damage to collagenous tissue was considered to be irreversible. However, it is now believed that disorders associated cartilage deterioration may be the result of a progressive decrease in the glycosaminoglycan (GAG) content of native cartilage. GAG's such as, for example, hyaluronic acid, proteoglycans, and glucosamines, are a group of natural compounds that form an integral part of the skin, cartilage, joints and other important tissues including many body fluids. These molecules exist as part of the extracellular matrix (ECM) and function as important morphogenic signaling molecules as they bind and present growth factors to immature cells. They play a role in cartilage development and repair and may contribute to the function of healthy joints. However, with age, synthesis of these molecules, particularly proteoglycans, begins to decrease causing the tissue to become dehydrated and brittle (De Groot J., et al Arth Rheum 1999 May: 42(5): 1003-009). For example, over time the molecular weight and concentration of proteoglycan responsible for maintenance of disc fluid content, begins to decrease leading to dehydration of the nucleus pulposus, and other deleterious changes which may negatively impact on a discs mechanical properties (Urban J. P., and J. F. McMullin, Spine Feb. 13, 1988, (2):179-87) Similarly, a decrease in the proteoglycan and hyaluronic acid content of articular cartilage with age leads to dehydration and brittleness. Friction begins to increase between opposing joint surfaces, which over time wears down the cartilage and leads to painful bone to bone contact. With age, both intervertebral discs and articular cartilage lose the ability to self-repair. Cumulative damage often leads to severe debilitation of the individual. If the mechanical function of the cartilage can be restored by replacing it with an allograft having normal properties, or otherwise supplementing the GAG content of the disc or joint cavity, some of these problems will be eliminated and others may be alleviated with surgeries that are not as severe as spinal fusion or joint replacement. This may accomplished through direct transplantation of a healthy disc, a portion of a healthy disc, or through infusion into the tissue of those extracellular matrix molecules and cells normally found in healthy mature cartilage.

Over time, the composition of the extracellular matrix of a disc changes as native cells of the nucleus pulposus either alter their phenotype or are replaced by cells that invade from other areas. These alterations result in changes in the biochemical activity of the ECM that leads to directly or indirectly to deterioration of the disc. For example, a decrease in notochordal and nucleus pulposus cells responsible for regulating proteoglycan synthesis leads directly to dehydration of the disc. (Aguiar, D. J. et al Exp Cell Res 1999 (246):129-137). Okum, M. et al J. Orthop. Res 1997 (15):528-538 demonstrated that gene expression in type II collagen cells was upregulated following experimentally induced degeneration of rabbit discs, causing changes in tissue composition which indirectly result in damage to the disc. Thus, the cellular composition and activity of the extracellular matrix is critical to maintaining healthy intervertebral discs.

The increase in surgical treatments conducted to repair damaged intervertebral discs is staggering. From 1979 to 1990, spinal surgeries increased in the United States by 137 percent. In 1997 more than 213,000 spinal fusion procedures were performed in the United States alone (National Institute of Health, 1997 Vital Statistics). However, for most patients these procedure are inadequate because they either eliminate pain without restoring function to the disc, or fail to preserve spinal mobility which often leads to degeneration of adjacent discs. Worldwide, the prosthetic disc replacement market has been estimated at over $2 billion annually (Med Tech Insight, February 2000). However, these discs are primarily composed of inert, polymeric substances, incapable of interacting with native cells and thus preclude natural recovery from subsequent damage. Therefore, a need remains in the field for methods and products capable of restoring natural mechanical and physical properties to a vertebral column through repair or replacement of a damaged intervertebral disc and methods and products, which enable the disc to self-repair. A similar need exists for methods and products to repair damage to collagenous tissue found in association with other articulating joints.

### BRIEF DESCRIPTION OF THE DRAWINGS

FIG. 1 shows a front view of one embodiment of an implant of the present invention prior to machining to create mechanical interlock. The dotted line represents vertebral bone to be removed during machining.

FIG. 2 shows a top view of one embodiment of the present invention showing a bone bridge machined to receiving an opposite machined vertebrae.

FIG. 3 shows a portion of one embodiment of the present invention to depict how the allogenic disc implant is received by an endogenous vertebrae.

FIG. 4 shows one embodiment of the present invention inserted between two adjacent vertebrae.

FIG. 5 is a front view of an intervertebral disc between upper and lower vertebrae showing the structure of a normal, healthy nucleus pulposus.

FIG. 6A is a front view an intervertebral disc between upper and lower vertebrae, with the nucleus pulposus removed.

FIG. 6B is a front view an intervertebral disc between upper and lower vertebrae, depicting the injection of material.

FIG. 6C is a front view an intervertebral disc between upper and lower vertebrae, after having been injected with material.

FIG. 7A is a front view of an intervertebral disc between upper and lower vertebrae, with a prolapsed disc.

FIG. 7B is a front view of an intervertebral disc between upper and lower vertebrae, with the prolapsed portion of the nucleus pulposus removed.

FIG. 7C is a front view of an intervertebral disc between upper and lower vertebrae, with the prolapsed portion of the nucleus pulposus removed, showing injection of material to replace section of the nucleus pulposus removed.

FIG. 7D is a front view of an intervertebral disc between upper and lower vertebrae, showing a nucleus pulposus after injection of material.

FIG. 8A is a front view of an intervertebral disc between upper and lower vertebrae, showing a degenerated disc with diminished disc height.

FIG. 8B is a front view of an intervertebral disc with diminished disc height disc between upper and lower vertebrae, and depicting injection of a material into a degenerated nucleus pulposus to restore disc height.

FIG. 8C is a front view of an intervertebral disc with disc height restored through injection of a material into the nucleus pulposus.

### SUMMARY OF THE INVENTION

The subject invention pertains to novel implants, and implant procedures that serve to restore the natural mechanical properties of cartilage and to provide an alternative surgical method for repair of cartilage found in association with joints. In one embodiment the subject method is less invasive than traditional repair procedures while in another embodiment the subject method avoids the deleterious side effects, such as, for example, increased stress and pain associated with degeneration of adjacent discs, or tissue rejection that sometimes accompany traditional procedures. The method and products may be adapted for use in treatment of all types collagenous tissue found in association with joints. According to one embodiment, a nucleus pulposus from an allograft or xenograft source is injected into a nucleus pulposus of a recipient in need. According to another embodiment, the nucleus pulposus of an aged and weakened vertebral disc is removed and at least one nucleus pulposus from an allogenic or xenogenic donor source is injected into the void created to thereby improve the mechanical function of the weakened disc. In another embodiment, disc stem/progenitor cells collected from healthy discs are cultured, grown and injected into the nucleus pulposus of a damaged disc in situ, or into a replacement nucleus pulposus prior to implantation. This technology may be used to completely replace a damaged nucleus pulposus with a healthy donor nucleus pulposus. According to another embodiment, an allograft human intervertebral disc with the upper and lower vertebrae still attached is harvested from a donor. The upper and lower vertebrae is machined in such a way as to provide a dovetail or other shape capable of forming a mechanical interlock with the patients own similarly prepared vertebrae. When implanted, the subject device provides renewed mobility to a spinal column through replacement of one or more damaged intervertebral discs.

In yet another embodiment, natural or synthetic materials are injected into a disc to restore normal mechanical and physiological properties to a disc undergoing degenerative disc disease. Transplantation offers new approaches to the repair of disc herniation and degenerative disc disease.

These methods have a wide range of applications in human spine disease and injury, and may be modified for use in treatment of other articular joint disorders.

Accordingly, it is a principle object of the present invention to provide a method of enhancing the mechanical function of an intervertebral disc.

It is a further object of the present invention to provide a method of replacing a damaged nucleus pulposus in an intervertebral disc.

It is a further object of the present invention to provide a method of augmenting the extracellular matrix of a nucleus pulposus.

It is a further object of the present invention to provide a non-surgical method of repairing collagenous tissue.

It is a further object of the present invention to provide a non-surgical method of repairing an intervertebral disc.

It is a further object of the present invention to provide a method of repairing an intervertebral disc through injection of natural or synthetic materials.

It is a further object of the present invention to provide natural or synthetic materials for injection into an intervertebral disc to restore disc function.

It is yet a further object of this invention to provide a composition for use in the treatment of damaged collagenous tissue.

It is yet another object of this invention to provide an implant to restore normal mechanical function to a vertebral column.

It is still another object of the present invention to provide an implant, which integrates with the existing spinal column.

It is a further object of the present invention to provide a method for treating degenerative disc disease, which does not compromise the integrity of adjacent vertebrae.

Yet another object of the present invention is to provide a method for restoring normal function to a damaged vertebral column.

Other objects and advantages of this invention will become apparent from review of the complete disclosure and the claims appended to this disclosure.

### DESCRIPTION OF THE PREFERRED EMBODIMENTS

The subject invention is primarily intended as a preventative measure to the onslaught of problems brought about by degenerative disc disease (DDD). It is preferably intended to address disc problems that are being experienced by a patient before rupture or other extensive damage to the annulus fibrosus of the disc has occurred. Thus, the subject invention will provide its best benefits for the patient if implemented at the early or intermediate stage of DDD, because the presence of a competent annulus fibrosus is preferred. However, the present methods and products may also be used with patients who have experienced traumatic injury to a joint. Repair of other collagenous tissue found in association with articulating joints is also contemplated.

According to one embodiment, allogenic nucleus pulposus is removed, collected, and immediately implanted or preserved by appropriate means for later injection into the patient. The endogenous nucleus pulposus is preferably removed through irrigation and aspiration, which can be done using conventional medical equipment. The collected allogenic nucleus pulposus is then injected into the void created by removal of the endogenous nucleus pulposus. To facilitate healing and ultimately improve the clinical results of the procedure, one or more growth factors may be added to the allogenic and/or xenogenic nucleus pulposus. The term “growth factor” as used herein refers to a polynucleotide molecule, polypeptide molecule, or other related chemical agent that is capable of effectuating differentiation of cells. Examples of growth factors as contemplated for use in accord with the teachings herein include an epidermal growth factor (EGF), transforming growth factor-alpha (TGF-α), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), and/or platelet derived growth factor (PDGF). Growth factors for use in accord with the teachings herein can be extracted from allograft, xenograft and/or autograft tissue, or can be produced by recombinant/genetic means, or be encoded by nucleic acids associated with appropriate transcriptional and translational elements. It will further be appreciated from the present disclosure that the implant may be contacted with cells prior to implantation. For example, human mesenchymal or other stem cells, such as those disclosed in any of U.S. Pat. Nos. 5,486,359; 5,811,094; 5,197,985; 5,591,625; 5,733,542; 5,736,396; 5,908,784; 5,942,255; 5,906,934; 5,827,735; 5,962,325; 5,902,741; 4,721,096; 4,963,489; (all of which are hereby incorporated by reference), may be contacted with, infused into or cultured on the implants of the present invention.

Alternatively, tissue biopsies taken from the patient, may serve as a source to harvest cells. Indigenous tissue cell populations are generated from cells extracted from the biopsied tissue for use as replacement cells. Tissue damaged as the result of degenerative disc disease is thereby minimizing the possibility of rejection. Alternatively, transplantation of syngeneic cells may be used to similarly reduce the likelihood that the implanted cells will be rejected by the recipient's body. Depending on the type of stem cells used and the particular requirement of the patient, the stem cells will be prepared and treated accordingly.

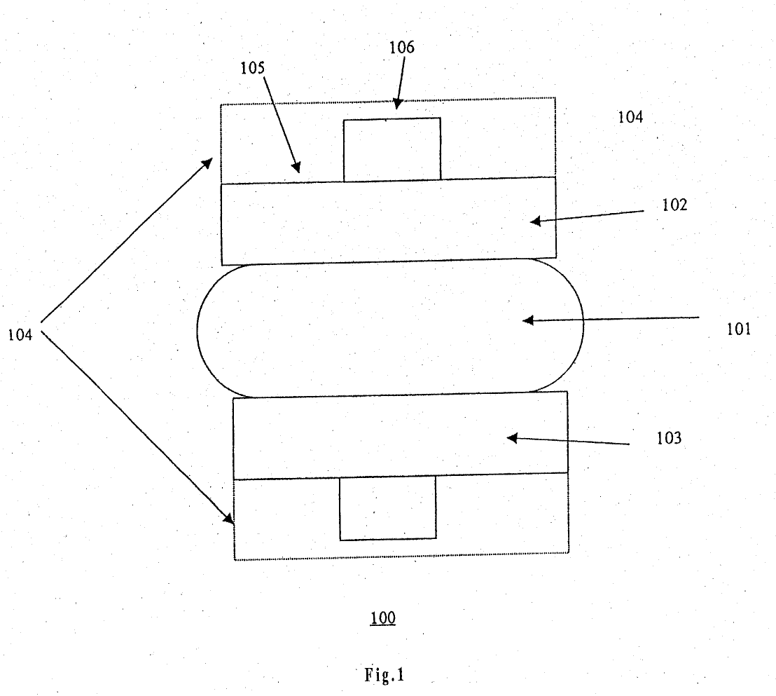

In an alternate embodiment, an implant is used to replace a damaged intervertebral disc. Accordingly, a physician will conduct an examination on a patient experiencing symptoms of disc trouble, or subjected to traumatic injury to diagnose and identify whether the patient has a ruptured, damaged or weakened intervertebral disc. Examination and tests typically involve the use of x-ray, MRI or other diagnostic imaging procedures. The location of a damaged disc is identified and noted according to the immediately adjacent vertebrae. For example, if a patient's has a damaged disc between cervical vertebrae number 5 (C5) and 6 (C6), a surgeon would identify the area above and below C5 and C6 as the site of incision. Through surgical techniques known in the art the damaged disc between C5 and C6 is removed. The surgeon then machines or carves slots into the bottom of C5 and the top of C6 in situ. An allograft implant comprising a healthy intervertebral disc attached C5 and C6 vertebrae is procured from a donor. A portion of the top of the donor's C5 vertebrae is cut to fit into the bottom of the patients C5 vertebrae, and the bottom of the donors C6 vertebrae is cut to fit into the top of the patients C6 vertebrae. For example, via an anterior approach, a portion of the top of the allograft C5 vertebrae is cut to form a protrusion that fits into the bottom of the patients C5 vertebrae, or vice versa, or the bottom of the allograft C6 vertebrae is cut to form a protrusion that fits into the top of the patients C6 vertebrae, or vice versa. The donor and recipient vertebrae are machined such that when placed together they are capable of interlocking. Preferably, the respective vertebrae are machined to form a bone bridge design as shown in FIG. 1. Alternatively, vertebrae may be crafted to form respective ends of a dovetail interlock, a keyhole interlock, tongue and groove, and the like. Thus, upon implantation a patient's upper vertebrae is attached to the donor upper vertebrae segment, the allogenic intervertebral disc, attached to the donor upper and lower vertebrae, is positioned into the cavity created from removal of the endogenous disc, and the donor lower vertebrae is attached to the patient's lower vertebrae. In this way, a damaged disc is replaced with a healthy, normal intervertebral disc. Techniques known in the art for removing portions of intervertebral discs and implantation of spinal fusion devices are readily adapted to carry out the removal of the damaged disc, carving of the slots and implantation of a machined allograft disc in accord with the teachings herein. Examples of such procedures are set forth in U.S. Pat. Nos. 6,245,072; 6,004,326; and 6,096,080, incorporated herein by reference. Preferably, when implanting a whole, allograft intervertebral disc, the disc is inserted via an anterior approach. As the connected vertebrae fuse and heal over time, normal spinal mobility is regained. This provides a significant advantage over other methods, which remove a damaged disc and subsequently fuse the adjacent vertebrae. The present method allows for restoration of normal mechanical function of the spinal columns without causing damage to adjacent disc as is commonly observed for other disc replacement surgeries.

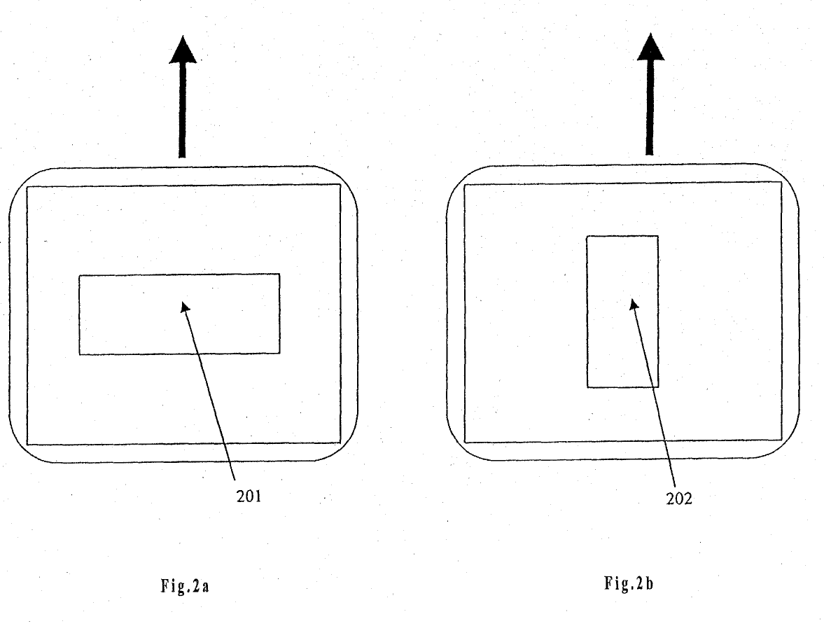

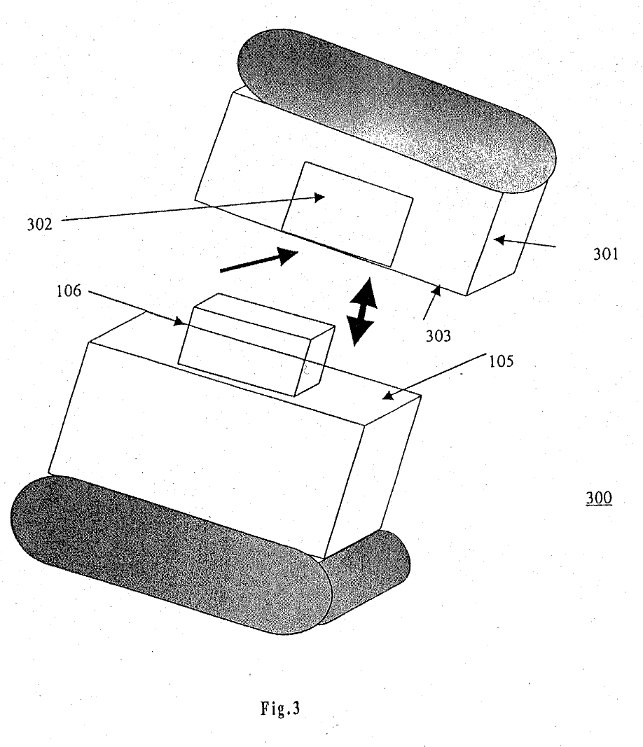

FIG. 1 shows a front view of one embodiment of the present invention generally represented at 100. The implant comprises an intervertebral disc 101 attached to an upper vertebrae 102 and a lower vertebrae 103. The vertebrae and disc are extracted intact from a donor. The upper 102 and lower vertebrae 103 are subsequently machined to create one end of mechanical interlock to hold the implant once implanted. A dotted line 104 generally represents the portion of the vertebrae that will be machined. In a preferred embodiment the vertebrae are machined to produce respective ends of a bone bridge design as shown, any design which forms a mechanical interlock and capable of supporting the forces associated with spinal movement is contemplated herein. As depicted, a bone bridge design is created such that a lower support 105 and upper support 106 are created. This design maximizes surface area available to support forces placed on the vertebrae, while providing a mechanical interlock mechanism to insure structural stability upon implantation. FIGS. 2a and 2b show top views of one embodiment depicting a intervertebral disc situated below a vertebrae that has been machined to create a mechanical interlock. The bone bridge may be designed to have an upper support running medial to lateral or anterior to posterior, the direction being dictated by the particular surgical procedure. FIG. 2a shows a portion of an implant with a vertebrae machined to have a medial to lateral upper support 201 (arrow represents the front of the body into which the implant is placed) FIG. 2b shows a portion of an implant with a vertebrae machined to have an anterior to posterior upper support 202. FIG. 3 shows an upper portion of one embodiment of the present invention to display the manner of implantation. In use, the implant (100 see FIG. 1) is inserted into a spinal column that has had a damaged intervertebral disc removed and wherein the remaining vertebrae have been machined to receive the implant. As shown, a patient vertebrae generally shown at 300 machined following extraction of the intervertebral disc which it previously covered. The vertebrae 301 has a receiving cavity 302 machined in it to receive the upper support 106 of the implant vertebrae. The side lateral edges 303 of the patient vertebral disc will rest on top of the lower support 105. In any embodiment, the implant and patient vertebrae are machined such that the implant can slide into the vertebrae and lock in place. Once the implant is in place between upper and lower patient vertebrae, it may be advantageous to secure the implant with known methods of temporary bone fixation.

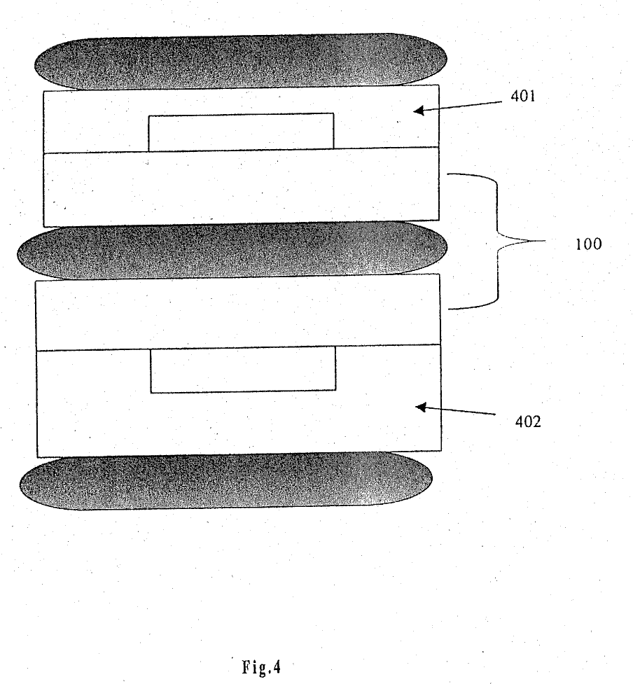

FIG. 4 shows a front view of an implant as it would exist once implanted between an upper patient vertebrae 401 and a lower patient vertebrae 402. As shown, once implanted the present invention provides a normal structure to a spinal column to restore normal mechanical function and mobility without the need to resort to spinal fusion techniques. It should be noted that the implant of the present invention may be adapted for use with any animal having a vertebral column, so long as the implant is procured from a species identical to that into which it will be placed. Thus, the present invention has wide ranging applications in the fields of human and veterinary medicine.

Upon extracting an implant from a donor, it may be implanted immediately or collected and kept frozen or preserved by other appropriate means such as by freeze-drying for later insertion into a patient. Furthermore, the implant may be treated to decellularize and inactivate any pathogens that might be present in the implant, as well as treating the implant to reduce antigenicity. Methods for treating the implant include those described in WO 00/29037 and WO 01/08715 A1, incorporated herein by reference. Following such treatments the allograft disc implant may be freeze-dried. To facilitate healing and ultimately improving the clinical result of the procedure, an osteoinductive composition (such as that described in WO99/38543) comprising DBM and/or one or more growth factors or cells can be coated or infused into the implant prior to implantation to speed recovery. Examples of such factors include epidermal growth factor (EGF), transforming growth factor-alpha (TGF-α), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), or platelet derived growth factor (PDGF), or like growth factors.

For patients experiencing symptoms of disc trouble, physicians will conduct examination and testing to diagnose and identify whether the patient has a ruptured, damaged or weakened intervertebral disc. Examination and tests typically involve the use of x-ray, CT scan, MRI or other diagnostic imaging procedures. Upon diagnosis of early or intermediate DDD, the site of need is accessed to determine which of the previously described procedures are appropriate for the patient. As one goal of the subject invention is to minimize the trauma associated with the procedure, it is preferred to access the site through an arthroscopic procedure or other technology that involves minimal invasion to the healthy portions of the disc and surrounding tissues. Where invasive surgery is required, such as for example, in transplantation surgery, the tissue transplanted is preferably treated with growth factors previously described to expedite healing.

In yet another embodiment of the present invention, a patients disc in need is subjected to injection or insertion of materials heretofore employed in soft-tissue augmentation therapies. Numerous biologic and synthetic materials are contemplated for injection into a nucleus pulposus to restore normal mechanical and or physiological properties to a damaged intervertbral disc. For example, one or more natural or synthetic glycosaminoglycans (mucopolysaccharides), such as, for example, hyaluronic acid (HA), chondroitan sulfate, dermatan sulfate, keratin sulfate, heparin, heparin sulfate, galactosaminoglycuronglycan sulfate (GGGS), and others, including their physiological salts, may be injected directly into the nucleus pulposus. Numerous studies have indicated that viscosupplementation with these materials may have therapeutic value. Injection of hyaluronic acid (HA) into a joint, for example, is known to improve the elasticity and viscosity of the synovial fluid, which in-turn increases joint lubrication and thereby decreasing joint pain. It has been suggested that HA plays a role in the stimulation of endogenous HA synthesis by synovial cells and proteoglycan synthesis by chondrocytes, inhibits the release of chondrodegradative enzymes, and acts as a scavenger of oxygen free radicals known to play part in cartilage deterioration. However, the benefits of injecting such materials either alone or in combination with other materials has not heretofore been realized. The inventors are unaware of any reference which teaches that such materials would be appropriate for injection into an intervertebral disc. Perhaps one reasons for this is the lack of methods for injection. Chondroitin sulfate and glucosamine injectables have similarly been shown to block the progression of articular cartilage degeneration. Arguably, other GAG's may provide similar protective or restorative properties having therapeutic value making them ideal candidates for injection into a disc undergoing degenerative disc disease. Another valuable property of GAG's is their strong ability to attract and retain water. Thus, it may be appropriate to mix GAG's with water or other aqueous materials to form a viscous gel that may then be injected into the space created from aspiration of a nucleus pulposus, or alternatively, added to an existing nucleus pulposus as a supplement. Natural “hydrogels” can thereby be formed which are capable of filling space in three dimensions and acting like packing materials that resist crushing and enable a disc to adequately absorb the shock associated with movement. It is submitted here by the inventors that through injection of one or more GAG's into a disc, direct anabolic stimulation of process associated with cartilage repair and development, proteoglycan synthesis and other processes contributing to healthy disc anatomy and physiology will be fostered, while degrading catabolic process, such as, for example, matrix metalloproteinase activity known to decreases proteoglycan content, will be reduced or eliminated. Proteoglycans, particularly aggrecan, play an important physiochemical role in the maintenance of disc hydration and morphology and may also be injected directly into a disc. Antioxidants having known chondroprotective abilities are also candidates for injection into the nucleus pulposus. Examples of these include tocophereol(vitamin E), superoxide dismutase (SOD), ascorbate (vitamin C), catalase and others. Further, amphiphilic derivatives of sodium alginate and the like are also contemplated herein for injection.

Other commercially available products thought to have beneficial properties also fall within the scope of this disclosure as possible components of the subject compositions. These include, for example, Zyderm® and Zyplast® (Collagen Co., Palo Alto, Calif.), (Mentor Corp., Goleta, Ga.), Dermalogen® (Collagenesis Inc., Beverly, Mass.), and Alloderm® (Life Cell Corp., Branchburg, N.J.) Autologous materials such as Isologen (Isologen Technologies, Inc., Paramus, N.J.) are also contemplated herein for injection. Additionally recombinant osteogenic protein-1 (OP-1) is a good candidate for injection because of its ability to promote the formation of a proteoglycan rich matrix by nucleus pulposus and annulus fibrosus cells.

Phospholipid transfer is also contemplated herein for repair of an intervertebral disc, and/or as a component of the subject injectables to treat other joints including, but not limited to, the knee, hip and shoulder. Autologous fat transplantation has been conducted for years in the field of soft tissue augmentation. Liposuctioned fat has been isolated from areas such as the abdomen, buttocks, thighs and other areas having a high fat concentration and injected into another area to alter the shape of a tissue, such as, for example, cheek augmentation. Recent studies suggest that phospholipids may play a critical role in joint function. Data published by Hills et al. (Br. J. Rheumatol February 1998; 37(2):137-42 suggests that the phospholipid present in synovial fluid may be the component that plays the greatest role as a load-bearing lubricant in joints (on the articular surface). If true, then administration of Hyaluronic acid (HA) alone to a joint or synovial environment will not have as great a lubricating activity as formulations including phopholipids. Thus, autogenic, allogenic or xenogenic fat liposuctioned or otherwise extracted from muscle or other tissue can be treated to isolate phospholipids, which then may be used to aid repair of a damaged joint. In a preferred method, extracted tissue is treated with organic solvents such as, for example, Chloroform and/or alcohols such as, for example, Methanol to isolate phospholipids from extraneous tissue. The solvent and/or alcohol is then removed through evaporation. The phospholipid residue remaining is added to sterile solutions which is subsequently injected into a joint capsule to aid in the lubrication. Phospholipids can also be combined with Hyaluronic acid (Hymedica) to further enhance the lubricating activity since it is believed that HA serves as a water-soluble proteinaceous carrier for phospholipids. It is submitted here by the inventors that injection of phospholipids alone or in combination with HA or other GAG into a joint will help restore healthy function to a damaged joint. Additionally, injection of such compositions into a damaged intervertebral disc may aid volume augmentation and help restore normal biomechanical function to the disc. Allogenic and xenogenic fat tissue may be also be used if the tissue is properly treated prior to injection.

In another embodiment, a composition comprising ground annulus fibrosus mixed with nucleus pulposus materials may be injected into a damaged disc to aid repair. Preferably, this material is obtained through processing of a donated intervertebral disc. In one embodiment, the tough annulus fibrosus material is ground to a particle form and mixed with the viscous nucleus pulposus material to create an injectable gel. It is submitted here by the inventors that matrix materials present in the donor nucleus pulposus along with structural material found in the annulus fibrosus, when injected into a damaged disc, will cause direct stimulation of the natural repair process and aid disc repair and or regeneration.

Use of synthetic injectables is also contemplated. These are particularly applicable to situations where the primary goal is to restore bio-mechanical function to a disc. Examples of injectable synthetic materials that may be used include medical grade silicone, Bioplastique® (solid silicone particles suspended in polyvinylpyrrolidone carrier; Uroplasty BV, Netherlands), Arteplast®(microspheres of polymethylmethacrylate (PMMA)suspended in gelatin carrier; Artcs Medical, USA), Artecoll® ((smooth PMMA spheres suspended in bovine cartilage carrier; Artepharma Pharmazeu Tische, GMBH Co., Germany). Synthetic, non-animal derived hyaluronic gels such as, for example, Restylane® (Q-Med Aktiebolag Co., Sweden) may also be used. Further, synthetic hydrogel compositions may be employed as a filler material to restore normal shape to a disc, thereby restoring normal bio-mechanical functions.

Hyaluronic acid alone or in combination with other glycosaminoglycans may be used as a carrier to deliver a biologically active material. In a prefered embodiment, Hyaluronic acid and or other GAGs is used as a carrier for stem cells selected for and capable of differentiation into disc cells. Furthermore, various known or commercially available or yet developed hyaluronic acid and/or other GAGs can be used as a carrier, preservative or activator of stem cells to be implanted into or topically applied to a patient. The concentration and viscosity of the hyaluronic acid/GAG composition is routinely adjusted to suit a given purpose.

The phrase “biologically active materials” as used herein includes, but is not limited to proteoglycans, chondrocytes, fibroblasts, antimicrobials and/or antibiotics such as erythromycin, bacitracin, neomycin, penicillin, polymyxin B, tetracyclines, viomycin, chloromycetin and streptomycins, cefazolin, ampicillin, azactam, tobramycin, clindamycin and gentamycin, etc.; amino acids, magainins, peptides, vitamins, inorganic elements, co-factors for protein synthesis; hormones; endocrine tissue or tissue fragments; enzymes such as collagenase, peptidases, oxidases, etc.; polymer cell scaffolds with parenchymal or other cells; surface cell antigen eliminators; angiogenic or angiostatic drugs and polymeric carriers containing such drugs; collagen lattices; biocompatible surface active agents; antigenic agents; cytoskeletal agents; cartilage fragments, living cells such as chondrocytes, bone marrow cells, mesenchymal stem cells, natural extracts, tissue transplants, bioadhesives, growth factors, growth hormones such as somatotropin; bone digestors; antitumor agents; glycosaminoglycans, proteoglycans, fibronectin; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; nucleic acids; bioerodable polymers; epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF). The amounts of such medically useful substances can vary widely with optimum levels being readily determined in a specific case by routine experimentation.

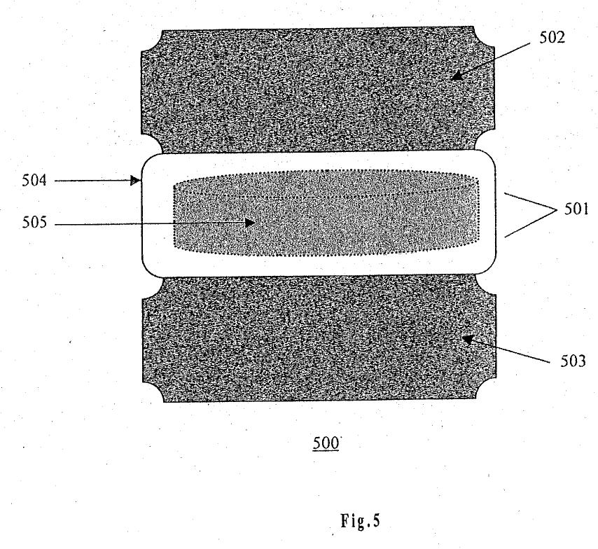

FIG. 5 depicts a front view of a healthy intervertebral disc complex as it would appear in situ, and generally indicated at 500, comprising an intervertebral disc 501 positioned between a superior vertebral body 502 and an inferior vertebral body 503. The disc 501 comprises an exterior annular fibrosus 504, which encapsulates an interior nucleus pulposus 505.

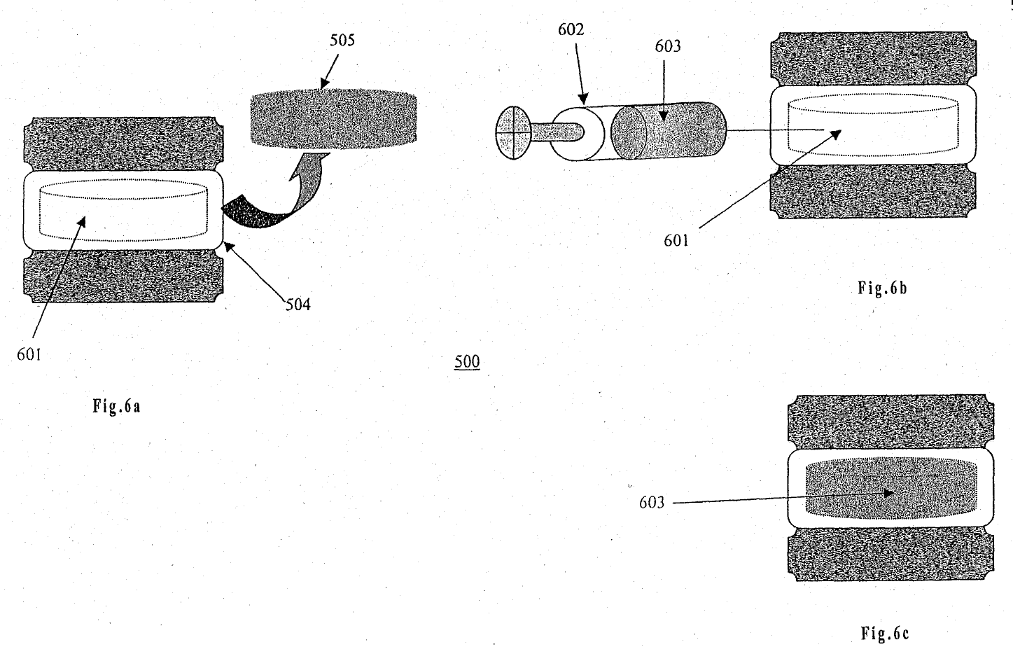

As previously described, over time structural and physiological changes may alter the composition of the disc which necessitates intervention. FIG. 6A depicts an intervertebral disc complex 500, wherein the nucleus pulposus 505 has been removed leaving a void 601 within the annulus fibrous 504. Preferably, the removal is achieved through aspiration, but other techniques known in the art for removal may be employed. FIG. 6B shows a syringe 602 containing natural or synthetic material 603 appropriate for injection into the void 601 to replace the extracted nucleus pulposus. Preferably, the material 603 used has a viscosity comparable to that of natural nucleus pulposus material, and which has a resiliency to withstand the force of compression associated with movement. Materials suitable for injection may be natural, synthetic or combinations of both so long as the material provides one or more properties useful in restoring some function to the disc. Thus, the exact composition of materials used will depend upon the desired result. For example, if the goal is to restore normal physiological properties to the disc over a long time frame, it would be beneficial to inject biological materials capable of promoting anabolic activities such as proteoglycan synthesis, cartilage formation or similar restorative functions. If however, the goal is to restore immediate bio-mechanical function to a disc, it may be appropriate to inject a synthetic polymer into the void 601. FIG. 6C shows an intervertebral disc complex 500 following injection of the material 603. Once injected, the material 603 preferably completely occupies the void 601 created, thereby restoring the normal disc structure.

Often, due to physiological changes in the disc composition, or through injury, a disc will “slip” or prolapse, such that a portion of the nucleus pulposus pushes against the annulus fibrosus to create a bulge. The protruding tissue may then press against adjacent nerves and cause severe pain.

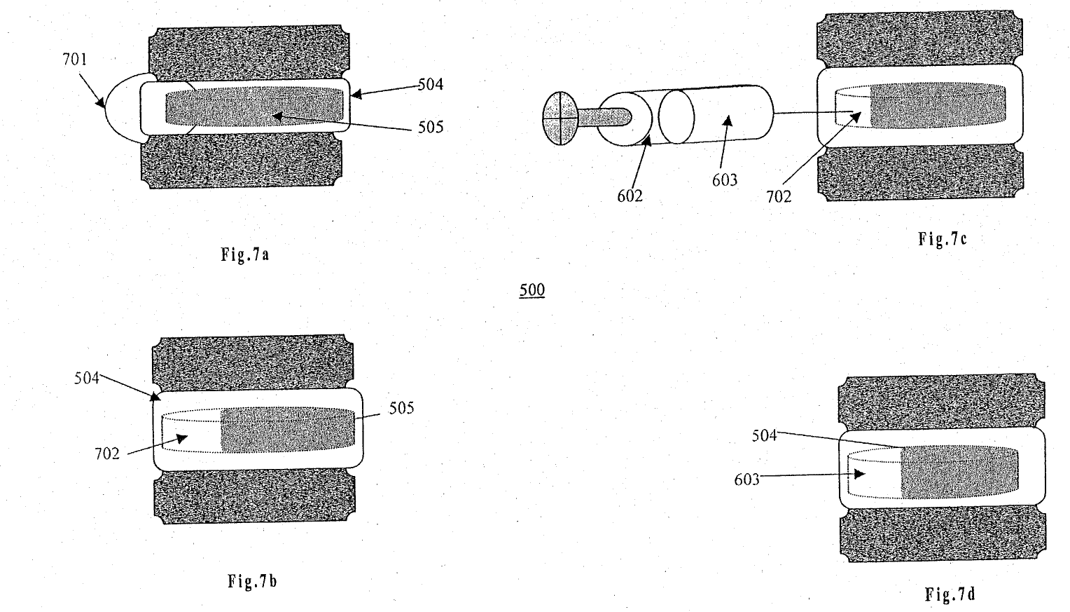

In these situations, removal of a portion of nucleus pulposus is indicated to relieve pressure on the walls of the annulus fibrosus. FIG. 7A depicts an intervertebral disc complex 500, wherein a migrating segment 506 of the nucleus pulposus 505 presses against the annulus fibrosus 504 causing a prolapse bulge 701 to develop. A variety of methods are known in the art, such as chemonucleolysis, to relieve the pressure placed on the walls of the annulus fibrosus, thereby reducing the expanse of the protruding tissue. FIG. 7B depicts an intervertebral disc complex 500, wherein the prolapse has been reduced by removal of the protruding portion of the nucleus pulposus 505 to create a chamber 702 within the annulus fibrosus 504. FIG. 7C depicts materials 603 within a syringe 602 being injected into the chamber 702. Ideally, because only a portion of the nucleus pulposus has been removed, the material 603 is combined with one or more biologic materials capable of augmenting the natural activities within the nucleus pulposus. FIG. 7D depicts a nucleus pulposus after injection of the material 603. Over time the injected materials 603 will integrate with the native nucleus pulposus 505 material to help restore normal physiological activities within the disc.

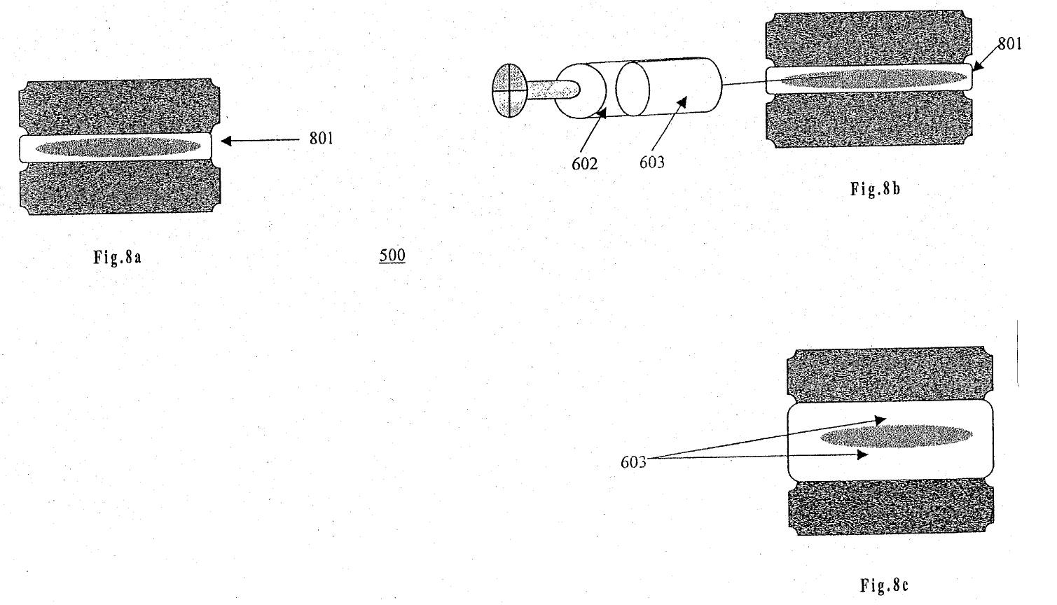

Another problem associated with degenerative disc disease is the gradual loss of fluid in the nucleus pulposus, causing disc depression which sets off a cascade of painful symptoms as the vertebral column attempts to adjust to this alteration in shape. FIG. 8A shows an intervertebral disc complex 500 having a depressed disc 801. FIG. 8B depicts a syringe 602 containing a material 603 that is injected into nucleus pulposus 505 of the depressed disc 801 to restore volume to the disc. FIG. 8C depicts a normally shaped intervertebral disc complex 500 resulting from injection of material 603.

The following examples are illustrative of the invention and are not meant to be limiting:

### EXAMPLE 1

### Replacement Of The Nucleus Pulposus

A patient presenting symptoms of degenerative disc disease was examined and the damaged disc was identified through MRI imaging. A 25 gauge needle with a 5 ml injector was inserted percutaneously into the damaged intervertebral disc and the nucleus pulposus was aspirated. A second identical procedure was conducted to obtain healthy, allogenic, cadaveric nucleus pulposus. The healthy nucleus pulposus was infused with growth factors and selected stem cells to help speed recovery, and then injected into the disc cavity to replace the endogenous nucleus pulposus extracted. Disc degeneration decreased following insertion of the healthy nucleus pulposus.

### EXAMPLE 2

### Injection Of Material Into The Cavity Created By Aspiration Of A Nucleus Puiposus

A patient presenting symptoms of degenerative disc disease is examined and the damaged disc is identified through MRI imaging. A 25 gauge needle with a 5 ml injector is inserted percutaneously into the damaged intervertebral disc and the nucleus pulposus was aspirated. A viscous formulation comprising natural hyaluronic acid and chondroitin sulfate is then injected into the disc cavity to replace the endogenous nucleus pulposus material extracted. In situ proteoglycan synthesis is expected following injection indicating that restoration of normal physiological processes is probable.

### EXAMPLE 3

### Augmentation Of Nucleus Pulposus Through Injection

A patient presenting symptoms of degenerative disc disease is examined and the damaged disc is identified through MRI imaging. A syringe is filled with a formulation comprising hyaluronic acid, chondroitan sulfate and the antioxidant ascorbate. A 25 gauge needle with a 5 ml injector is attached to the syringe and is inserted percutaneously into the damaged intervertebral disc directly into the nucleus pulposus. The injected material augments the present material by providing materials which help stop the catabolic degradation cascade associated with disc degeneration. In situ proteoglycan synthesis and reduction in the activity of matrix metalloproteinases is expected following injection indicating that restoration of normal physiological processes is probable.

### EXAMPLE 4

### Regeneration Of Disc Height Through Injection Of Material

A patient presenting symptoms of degenerative disc disease is examined and the damaged disc is identified through MRI imaging. Four syringes are filled with a mixture capable of restoring disc height and promote restoration. A first syringe contains ground annulus fibrosus (AF) mixed with viscous nucleus pulposus (NP). A second syringe contains a mixture comprising AF, NP and hyaluronic acid (HA). A third syringe contains a mixture comprising AF, NP, HA and a glycosaminoglycan (GAG). A fourth syringe contains a mixture comprising AF, NP and GAG. In each case, the syringe is attached to a 25 gauge needle with a 5 ml injector for insertion into the damaged intervertebral disc. Material contained within the syringe is then injected into the disc space and the volume of the nucleus pulposus is immediately increased in three dimensions. In each case, the viscous material injected provides similar mechanical properties to those associated with healthy nucleus pulposus material. The disc regains normal compressibility instantly indicating that restoration of normal mechanical properties are probable.

### EXAMPLE 5

### Disc Repair Using Implant

A patient presenting symptoms of degenerative disc disease is examined and the damaged disc is identified through MRI imaging. The location of the disc is identified according to the adjacent vertebrae. The site of extraction is marked and the mucosa is surgically cut and turned back to expose the vetebral column. In one example, cervical vertebrae 5 and 6 (C5 and C6) are identified as the vertebra immediately adjacent the damaged disc. The damaged endogenous disc is then excised and the space between adjacent vertebrae is maintained through supports. The upper and lower vertebrae are then machined to form respective receiving ends of a bone bridge design. A second surgical procedure is conducted on a donor cadaver. The vertebral column of the donor is exposed at a site corresponding to the donor C5 and C6 vertebrae. A tissue sample comprising the C5 and C6 vertebrae having a healthy intervertebral disc still attached is excised from the donor. The donor C5 and C6 vertebrae are then machined to form the respective insertion ends of a bone bridge design. The tissue is then implanted into the patient such that the C6 donor vertebrae interlocks with the C6 patient vertebrae, while the C5 donor vertebrae interlocks with the C5 patient vertebrae. The healthy disc between donor C5 and C6 is received into the chamber created between patient C5 and C6 from removal of the endogenous disc. Following implantation the interlocking vertebrae fuse and bone remodels, and the healthy disc adequately supports forces placed thereon, thereby restoring the normal mechanical function to the vertebral column.

Other modifications of the above-described invention are envisioned. For example, molecular combinations for use in treatment of collagenous tissue damage is contemplated. Molecular and genetic characterization studies designed to recognize known transcripts for ECM genes (e.g. tenascin, proteoglycans) as well as the discovery of novel morphogenic proteins is also contemplated. Further, use of the present methods and compositions in treatment of damage to articular cartilage is also contemplated. The disclosure of all references cited herein are incorporated by reference to the extent they are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method of enhancing the mechanical function of an intervertebral disc of a patient in need, said method comprising extracting at least one nucleus pulposus from an allogenic or xenogenic source, or both, and implanting said extracted nucleus pulposus into said patient at a site of need.

2. The method of claim 1, wherein said method further comprises removing an endogenous nucleus pulposus from said intervertebral disc of said patient thereby forming a void and injecting said extracted allogenic or xenogenic nucleus pulposus into said void.

3. The method of claim 1, wherein said method further comprises adding epidermal growth factor (EGF), transforming growth factor-alpha (TGF-α), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), or platelet derived growth factor (PDGF), or combinations thereof, to said extracted nucleus pulposus.

4. The method of claim 1, wherein said method further comprises adding stem cells, fibroblasts, muscle cells, or neuronal cells, or combinations thereof, to said extracted nucleus pulposus.

5. The method of claim 1, wherein extracting comprises aspirating nucleus pulposus from an allogenic intervertebral disc.

6. The method of claim 5, further comprising storing said aspirated nucleus pulposus for at least 24 hours before implanting said nucleus pulposus into said patient.

7. An extracted nucleus pulposus from an allogenic or xenogenic source for injection into a human intervertebral disc.

8. The extracted nucleus pulposus of claim 7, wherein said nucleus pulposus is supplemented with one or more growth factors, or one or more cells, including stem cells, one or more GAGs including hyaluronic acid, or combinations thereof.

9. A composition capable of restoring natural mechanical properties to an intervertebral disc undergoing degenerative disc disease comprising clonally expanded populations of stem cells.

10. The composition of claim 9, wherein said stem cells may be selected from the group comprising totipotent stem cells, pluripotent stem cells, multipotent stems cells and combinations thereof.

11. The composition of claim 9, wherein said stem cells are capable of differentiating in into chondroblasts, fibroblasts, secretory cells, mature notochord cells and combinations thereof.

12. The composition of claim 9, wherein said disc stem cells are capable of enriching the extracellular matrix of an intervertebral disc through production of growth factors, proteoglycans, glycosaminoglycans and combinations thereof.

13. The composition of claim 12, wherein said growth factors are selected from the group comprising peptide growth factors, epidermal growth factor (EGF), transforming growth factor-alpha (TGF-α), transforming growth factor-beta (TGF-β), human endothelial cell growth factor (ECGF), bone morphogenetic protein (BMP), fibroblast growth factor (FGF), insulin-like growth factor (IGF), cartilage derived morphogenetic protein (CDMP), platelet derived growth factor (PDGF), and combinations thereof.

14. The composition of claim 12, wherein said glycosaminoglycans are selected from the group comprising hyaluronic acid, chondroitin sulfate, dermatan sulfate, keratin sulfate, heparin, heparin sulfate, their physiological salts, or combinations thereof.

15. The composition of claim 12, wherein injection of said composition into an intervertebral disc is useful in preventing, inhibiting and reversing the affects of degenerative disc disease.

16. A composition to treat joint disease comprising stem cells in a carrier comprising one or more glycosaminoglycans.

17. The composition of claim 16, wherein said joint is selected from group consisting of cartilaginous and synovial joints.

18. The composition of claim 17, wherein said joint is selected from the group comprising amphiarthroidal joint, ball and socket joint, condyloid joint, ellipsoid joint, saddle joint, hinge joint or pivot joint.

19. The composition of claim 16, wherein said stem cells are selected from the group comprising totipotent stem cells, pluripotent stem cells, multipotent stems cells and combinations thereof

20. The composition of claim 16, wherein said one or more glycosaminoglycans is selected from the group comprising hyaluronic acid, chondroitin sulfate, dermatan sulfate, keratin sulfate, heparin, heparin sulfate, galactosaminoglycuronglycan sulfate their physiological salts, or combinations thereof.

21. An improved method of treating an intervertebral disc undergoing degenerative disc disease, wherein a solution capable of restoring the natural mechanical functions of a damaged disc is injected into a disc, the improvement consisting essentially of injection into the damaged disc of a population of stem cells capable of restoring normal function to the disc by enriching extracellular matrix of the disc through production of glycosaminglycan and growth factors.

22. An implant comprising an intervertebral disc attached to an upper and lower vertebra, wherein said upper and lower vertebrae are machined to provide a mechanical interlock between said implant vertebrae and a corresponding vertebral body in situ.

23. The implant of claim 22, wherein said implant is adapted to be received into a vertebral column of a patient.

24. The implant of claim 22, wherein said implant is extracted from an allogenic or xenogenic source.

25. The implant of claim 22, wherein said implant restores mobility to a spine without damaging adjacent vertebrae.

26. The implant of claim 22, wherein said implant is designed to withstand normal mechanical stress placed on said vertebral column.

27. The implant of claim 22, wherein said implant is machined to form one end of an interlocking design selected from the group comprising dove tail, tongue and groove, key hole, bone bridge and combinations thereof.

28. A method for repairing a damaged vertebral column in a patient comprising:
a) identifying the location of a damaged disc;
b) extracting said damaged disc;
c) procuring an implant comprising an intervertebral disc attached to an upper and a lower vertebra, said implant extracted from an allogenic or xenogenic source; and
d) machining said vertebrae of said implant and said vertebrae of said patient, such that said implant vertebrae and said vertebra of said patient are designed to be secured together.

29. The method of claim 28, further comprising inserting said implant into said vertebral column of said patient, wherein said healthy disc is positioned between said connected vertebrae.

30. The method of claim 28, further comprising locating the site of extraction and implantation, surgically cutting the mucosa at said area and turning back the adjacent tissue exposing the vertebral column.

31. The method of claim 28, wherein said machining of said implant vertebrae produces a first portion of a mechanical interlock.

32. The method of claim 28, wherein said machining of said patient vertebrae in situ produces a second, complimentary portion of said mechanical interlock.

33. The method of claim 32, wherein said first portion and said second portion are machined to form a mechanical interlock design selected from the group comprising tongue and groove, dove tail, bone bridge, keyhole, and combinations thereof.

34. The method of claim 33, wherein said implant interfaces with a patient's vertebral column through connection of said first and said second portions of said mechanical interlock.

35. An method of claim 34, wherein said implant is treated with a medically useful substance.

36. The method of claim 35, wherein said medically useful substance comprises epidermal growth factor (EGF), transforming growth factor-alpha (TGF-α), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), or platelet derived growth factor (PDGF), or combinations thereof.

37. The method of claim 28, further comprising storing said implant for at least 24 hours before implanting said implant into said patient.

38. A method of enhancing the function of an intervertebral disc of a patient in need, said method comprising injecting a chondroprotective material into a patient at a site of need.

39. The method of claim 38, wherein said chondroprotective material is selected from the group comprising glycosaminoglycans, including hyaluronic acid, ground annulus fibrosus, nucleus pulposus, proteoglycans, antioxidants, amphiphillic derivatives of sodium alginate, recombinant osteogenic protein-1 (OP-1), phospholipids, Zyderm®, Zyplast®, Fibrel, Dermalogen®, Micronized Alloderm®, Isologen, and combinations thereof.

40. The method of claim 38, wherein injection of said chondroprotective material inhibits or reverses the affects of degenerative disc disease.

41. The method of claim 38, wherein said chondroprotective material is derived from autogenic sources, allogenic sources, xenogenic sources, or combinations thereof.

42. The method of claim 38, wherein said chondroprotective material is selected from the group comprising medical grade silicone, hydrogels, GAG's, Bioplastique, Arteplast®, Artecoll®, Restylane®, and combinations thereof.

43. The method of claim 38, wherein injection of said chondroprotective material restores normal biomechanical function to a disc undergoing degenerative disc disease.

44. A method of repairing a prolapsed intervertebral disc comprising dissolution of prolapsed material followed by injection of an amount of chondroprotective material, proteoglycan synthesizing material, filler material or combinations thereof sufficient to restore normal structure to said disc.

45. The method of claim 44, wherein injection of said chondroprotective material restores normal disc height to a disc undergoing degenerative disc disease.

46. The method of claim 44, wherein one or more of said chondroprotective materials is injected into a nucleus pulposus to treat degenerative disc disease.

47. A method of restoring normal properties to a damaged intervertebral disc comprising the steps of injecting a composition comprising at least one injectable chondroprotective material and, optionally, at least one biologically active material into a patient at a site of need.

48. The method of claim 47, wherein said chondroprotective material is selected from the group comprising natural or synthetic hyaluronic acid, chondroitin sulfate, dermatan sulfate, keratin sulfate, heparin, heparin sulfate, galactosaminoglycuronglycan sulfate, their physiological salts or combinations thereof.

49. The method of claim 47, wherein said biologically active material is selected from the group comprising proteoglycans, glycosaminoglycans, chondrocytes, fibroblasts, hormones, collagen, cartilage fragments, mesenchymal stem cells, growth hormones; fibronectin; nucleic acids; epidermal growth factor (EGF), transforming growth factor-alpha (TGF-alpha), transforming growth factor-beta (TGF-beta), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), and/or platelet derived growth factor (PDGF), or combinations thereof.

50. A composition for injection into a spine comprising ground allogenic or xenogenic annulus fibrosus.

51. The composition of claim 50 further comprising allogenic or xenogenic nucleus pulposus.

52. A composition for treatment of a joint comprising autogenic, allogenic, or xenogenic phospholipids, or combinations thereof.

53. The composition of claim 52 further comprising material selected from the group comprising natural or synthetic hyaluronic acid, chondroitin sulfate, dermatan sulfate, keratin sulfate, heparin, heparin sulfate, galactosaminoglycuronglycan sulfate, their physiological salts or combinations thereof.

54. A method of treating a joint comprising injecting the composition of claim 53 into said joint.

55. A composition comprising stem cells in a carrier, wherein said carrier is a material selected from the group comprising natural or synthetic hyaluronic acid, chondroitin sulfate, dermatan sulfate, keratin sulfate, heparin, heparin sulfate, galactosaminoglycuronglycan sulfate, their physiological salts or combinations thereof.

56. The composition of claim 55, wherein said carrier is natural or synthetic hyaluronic acid, chondroitin sulfate, or a combination thereof.

57. A method of storing, preserving or stimulating stem cells comprising contacting said stem cells with a material selected from the group comprising natural or synthetic hyaluronic acid, chondroitin sulfate, dermatan sulfate, keratin sulfate, heparin, heparin sulfate, galactosaminoglycuronglycan sulfate, their physiological salts or combinations thereof.
